(19) United States
(12) Patent Application Publication
(10) Pub. No.: US 2006/0292072 A1
(43) Pub. Date: Dec. 28, 2006

(54) Agents for neutron capture therapy

(75) Inventors: Tarak D. Mody, Sunnyvale, CA (US); Jonathan L. Sessler, Austin, TX (US); Stuart W. Young, Portola Valley, CA (US)

Correspondence Address: WILSON SONSINI GOODRICH & ROSATI, 650 PAGE MILL ROAD, PALO ALTO, CA, 94304-1050, US

(73) Assignee: Pharmacyclics, Inc., Sunnyvale, CA (US)

(21) Appl. No.: 11/134,840

(22) Filed: May 19, 2005

Related U.S. Application Data
(63) Continuation-in-part of application No. 10/362,964, filed on Feb. 25, 2003, now abandoned.

(30) Foreign Application Priority Data
Aug. 28, 2001 (WO) PCT/US01/26773

(51) Int. Cl. A61K 51/00; C07F 5/00; A61K 31/555; C07D 257/02

(52) U.S. Cl. 424/1.11; 514/185; 534/15; 540/474

(57) ABSTRACT

Compounds, pharmaceutical formulations and methods for use in neutron capture therapy are provided, useful for treating diseases characterized by neoplastic tissue and arteriosclerosis.

### CLAIM OF PRIORITY

This application claims the benefit of priority from U.S. patent application Ser. No. 10/362,964, filed Feb. 25, 2003, which claims priority from International Patent Application PCT/US01/26773, filed Aug. 28, 2001, which claims the benefit of priority from U.S. Provisional Application No. 60/229,366, filed Aug. 30, 2000, both of which are incorporated herein, by reference, in their entirety.

### FIELD OF THE INVENTION

The present invention relates to the use of metal complexes, in particular metallotexaphyrins, in neutron capture therapy. Neutron capture therapy is useful in the treatment of diseases characterized by neoplastic tissue, such as tumors, or plaque caused by atherosclerosis or other atheromatous diseases. The invention also relates to novel metallotexaphyrins, and pharmaceutical compositions containing such compounds.

### BACKGROUND INFORMATION

A much sought-after goal with respect to the treatment of cancer is the development of a therapy that selectively destroys diseased tissue, while not adversely impacting healthy tissues. Some progress has been made toward such a goal, and has led, for example, to the discovery and use of the class of agents known as sensitizers. Sensitizers are selectively taken up by a tumor or plaque, which is then treated with one or more forms of energy, such as light, radiation, or sonic energy, or alternatively with a chemotherapeutic drug, in order to destroy the tumor or plaque.

Another known method of treating tumors employs neutron capture therapy (NCT), which has been used for treating brain tumors. NCT comprises a two-step process, each step of which when taken by itself has relatively little effect on normal cells; it is only when the two steps are combined that action is induced. Ideally, the two steps are 1) administration of a non-toxic neutron capture agent to a patient so as to provide selective uptake and/or retention of the agent within a tumor, followed by 2) irradiation of the site at which the neutron capture agent is retained with a neutron beam. The thermal (or slow) neutrons that are employed in such treatment cause little damage to normal tissue as compared to other types of radiation commonly used in the treatment of cancer, for example ionizing radiation such as protons, gamma rays, X-rays, and fast neutrons.

The measure of an atom's ability to capture neutrons is referred to as its “neutron capture cross-section”, measured in units of barns (1.0 b=10⁻²⁴ cm²), from which derives the sometimes more commonly used term “barns radius”. To varying degrees, all atoms have some ability to capture neutrons; for example, carbon, hydrogen and nitrogen have barns radii of 0.0035 b, 0.332 b and 1.9 b respectively. Therefore, it is clear that for a neutron capture agent to be effective in NCT, it must have the ability to capture neutrons much more efficiently (i.e., have a much higher barns radius) than those atoms that are normally found in cells, healthy or otherwise. Absent this property, neutron irradiation would lead to a low rate of capture of neutrons, and lack of selectivity, thus rendering the treatment ineffective.

Much of the early NCT work has been performed using an isotope of the element boron, identified as ¹⁰B, which has a barns radius of 3,840 b. ¹⁰B has the ability to absorb (or capture) slow or “thermal” neutrons, and, when impacted by such neutrons, is converted to a higher isotope, ¹¹B, which immediately disintegrates into linear energy transfer fission products, such as ⁷Li and high energy α-particles, which have the potential for destroying a cell and/or surrounding tissue.

Thus, to some degree ¹⁰B meets one of the requirements for a neutron capture agent (larger barns radius). However, a second requirement for a neutron capture agent is that it must selectively accumulate in the disease tissue (tumor cells, for example), while at the same time being readily cleared from normal tissue and the bloodstream. Absent such an effect, the neutron capture agent will also be distributed in normal tissue and blood, which will, when irradiated, be destroyed in the same manner as the tumor or plaque. Unfortunately, ¹⁰B does not meet this requirement, as it is not itself selective for tumor tissue. In an attempt to overcome this deficiency, ¹⁰B has been derivatized with certain agents with a view toward generating boron compounds that are selectively transported to the target tissue. For example, p-boron-phenylalanine has been used in the treatment of melonamas by NCT. However, this approach has not been entirely successful, as this boron compound has limited selectivity for tumors, and also poor tumor/blood concentration ratios, which leads to vascular endothelial damage upon radiation, causing damage to the normal brain.

Porphyrins and porphyrins-like compounds are known to accumulate in tumor cells. Accordingly, boron derivatives of such compounds have been prepared for testing as neutron capture agents in an attempt to overcome this disadvantage of low selectivity. For example, one such compound is a boronated porphyrin known as tetrakiscarborane carboxylate ester of 2,4-(a,b-dihydroxyethyl)deuteroporphyrin (BOPP; see, for example, J. Tibbitts, J. R. Fike, K. R. Lamborn, A. W. Bollen, and S. B. Kahl, Photochem. Photobiol., 69, 587 (1999). However, this compound has also been found to have poor selectivity for tumors, and additionally has a major limitation in that it is phototoxic.

Therefore, to remedy the disadvantages of existing boron compounds, elements other than boron were considered for neutron capture therapy. Gadolinium (Gd) is one such element, as it has a relatively large barns radius (48,800 b); notably, the ¹⁵⁷Gd isomer of gadolinium has a barns radius of 254,000 b. However, gadolinium itself poses the threat of ion toxicity, and the early gadolinium chelates prepared for use as MRI imaging agents were not selective for tumors and other targeted tissues. Additionally, the porphyrins that were suitable for the formation of boronated compounds do not form stable complexes with Gd, as in general these and other porphyrins do not possess central coordinating cores that are large enough to accommodate a large cation Gd(III). They have, therefore, been found unsuitable for use as carrier molecules for Gd, even supposing that such porphyrins were selective for neoplastic tissue (see Lyon et al, Tissue Distribution and stability of Metalloporphyrin MRI Contrast Agents; Magnetic Resonance in Medicine 4, 24-33 (1987); Brugger, Evaluation of 157 Gadolinium as a neutron capture agent, Strathinger. Oncol., 165 (1989).

Two non-porphyrin Gd complexes that have been successfully prepared and tested as neutron capture agents are gadolinium diethylenetriamine-pentaacetic acid (Gd-DTPA, CAS No. 86050-77-3) and gadoteric acid (Gd-DOTA, CAS No. 138071-82-6). However, they were not found to be tumor selective, and large amounts of the drug had to be administered for neutron capture therapy to be effective, and thus in the course of treatment normal tissue is destroyed as well as the neoplastic tissue. Additionally, Gd-DTPA and Gd-DOTA have rapid clearance rates from the tumor, and therefore in order to be effective neutron irradiation must occur very soon after administration of either of these drugs. It has also been reported (Chem. Pharm. Bull., 48(7) 1034-1038 (2000)) that a carborane Gd¹⁵⁷ complex of DTPA has been prepared and evaluated as an imaging agent and potential NCT agent, but found that it was not selectively accumulated in tumor tissue.

Accordingly, it remains desired to provide a stable, non-toxic neutron capture agent that:

a) is selectively taken up by tumors or other targeted tissue;

b) is rapidly cleared from healthy tissue and the bloodstream, while being retained in the tumor or other targeted tissue for a therapeutically beneficial period; and

c) has a metal core that has a large barns radius, which, when impacted by slow or “thermal” neutrons, produces energy capable of destroying the targeted tissue;

It further remains desired to provide a delivery agent that is also a radiation sensitizer, which would augment the effects of any radiation produced during the neutron capture process. Such a compound would also have synergistic benefits with standard radiation therapy and conventional chemotherapeutic treatment (such as doxorubicin and bleomycin).

A class of compounds known to be particularly useful as sensitizers are those known as metallotexaphyrins, in particular gadolinium texaphyrins (for example, see list of patents and patent applications below). It has now been discovered that they are surprisingly adaptable for use as neutron capture therapy agents.

### SUMMARY OF THE INVENTION

Accordingly, in a first aspect, the present invention provides a method for treating a disease or condition in a mammal resulting from the presence of neoplastic tissue or plaque caused by atherosclerosis or other atheromatous diseases, which method comprises:
a) administering to a mammal in need of such treatment a therapeutically effective amount of a neutron capture agent of Formula I: 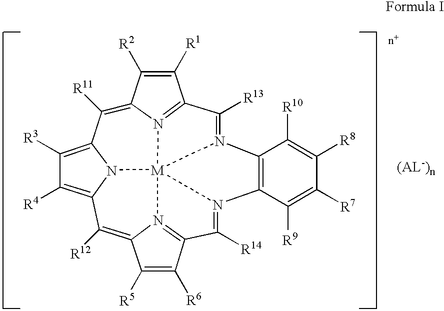
wherein:
AL is an apical ligand
M is a natural metal, an enriched metal, or a pure isotope thereof, having a neutron capture cross section greater than about 1,000 b;
n is an integer of 1-5;
R¹, R², R³, R⁴, R⁵, R⁶, R⁷, and R⁸ are independently chosen from the group consisting of hydrogen, halogen, hydroxyl, optionally substituted alkyl, optionally substituted alkenyl, optionally substituted alkynyl, optionally substituted aryl, optionally substituted heteroaryl, optionally substituted haloalkyl; nitro, acyl, optionally substituted alkoxy, saccharide, optionally substituted amino, carboxyl, optionally substituted carboxyalkyl, optionally substituted carboxyamide, optionally substituted carboxyamidealkyl, optionally substituted heterocycle, optionally substituted cycloalkyl, optionally substituted arylalkyl, optionally substituted heteroarylalkyl, optionally substituted heterocycloalkylalkyl, and the group —X—Y, in which X is a covalent bond Y is a catalytic group, a chemotherapeutic agent, or a site-directing molecule; and
R⁹, R¹⁰, R¹¹, R¹², R¹³ and R¹⁴ are independently hydrogen, optionally substituted alkyl, optionally substituted aryl, optionally substituted alkoxy, optionally substituted carboxyalkyl, or optionally substituted carboxyamidealkyl;
with the proviso that the halogen is other than iodide and the haloalkyl is other than iodoalkyl; and
   b) irradiating the neoplastic tissue or the atheroma with a neutron beam.

M can be a natural metal, for example Ca⁺², Mn⁺², Co⁺², Ni⁺², Zn⁺², Cd⁺², Hg⁺², Sm⁺², UO⁺², Mn⁺³, Co⁺³, Ni⁺³, Fe⁺³, Ho⁺³, Ce⁺³, Y⁺³, In ⁺³, Pr⁺³, Nd⁺³, Sm⁺³, Eu⁺³, Gd⁺³, Tb⁺³, Dy⁺³, Er⁺³, Tm⁺³, Yb⁺³, Lu⁺³, La⁺³ or U⁺³. Preferably, M is an enriched metal, more preferably selected from: enriched gadolinium, enriched cadmium, enriched europium, enriched mercury and enriched samarium. Particularly preferred are: ¹⁵⁵Gd- and/or ¹⁵⁷Gd-enriched gadolinium, ¹¹³Cd-enriched cadmium, ¹⁵¹Eu-enriched europium, ¹⁹⁹Hg-enriched mercury, and ¹⁴⁹Sm-enriched samarium.

Most preferred are those compounds of Formula I in which M is a pure isotope, preferably ¹⁵⁵Gd, ¹⁵⁷Gd, ¹¹³Cd, ¹⁵¹Eu, ¹⁹⁹Hg or ¹⁴⁹Sm, especially those compounds of Formula I where M is the pure ¹⁵⁷Gd isotope of gadolinium or ¹⁵⁷Gd-enriched gadolinium.

Preferred apical ligands are, for example, derived from carboxylates of sugar derivatives, such as gluconic acid or glucoronic acid, cholesterol derivatives such as cholic acid and deoxycholic acid, PEG acids, or carboxylic acid derivatives, such as formic acid, acetic acid, propionic acid, butyric acid, pentanoic acid, methylvaleric acid, glycolic acid, pyruvic acid, oxalic acid, malic acid, malonic acid, succinic acid, maleic acid, fumaric acid, tartaric acid, 3,6,9-trioxodecanoic acid, 3,6-dioxoheptanoic acid, 2,5-dioxoheptanoic acid, citric acid, benzoic acid, cinnamic acid, mandelic acid, methanesulfonic acid, ethanesulfonic acid, p-toluene-sulfonic acid, salicylic acid. Other preferred apical ligands include organophosphates, such as methylphosphonic acid and phenylphosphonic acid, phosphoric acid and other inorganic acids.

A second aspect of this invention relates to a method of using the compounds of Formula I in the treatment of a disease or condition in a mammal that results from the presence of neoplastic tissue, which method comprises the steps of administering to a such a mammal a therapeutically effective amount of a compound of Formula I, and 1) irradiating the area in proximity to the neoplastic tissue with neutrons, followed by 2) treating the area in proximity to the neoplastic tissue with a therapeutic energy means. Preferred therapeutic energies include photoirradiation, ionizing radiation, and ultrasound. Steps 1) and 2) may be reversed.

A third aspect relates to a method of using the compounds of Formula I in the treatment of a disease or condition in a mammal that results from the presence of neoplastic tissue or plaque caused by atheromatous disease, which method comprises the steps of administering to a such a mammal a therapeutically effective amount of a compound of Formula I, and a photosensitizer, such as Lu-Tex, and 1) irradiating the area in proximity to the neoplastic tissue with neutrons, followed by 2) treating the area in proximity to the neoplastic tissue or plaque with a therapeutic energy means, for example photoirradiation. Steps 1) and 2) may be reversed.

A fourth aspect relates to a method of using the compounds of Formula I in the treatment of a disease or condition in a mammal that results from the presence of neoplastic tissue, which method comprises the steps of administering to a such a mammal a therapeutically effective amount of a compound of Formula I, and 1) irradiating the area in proximity to the neoplastic tissue with neutrons, followed by 2) treating the area in proximity to the neoplastic tissue or plaque with a chemotherapeutic agent.

A fifth aspect of this invention relates to novel compounds of Formula I: 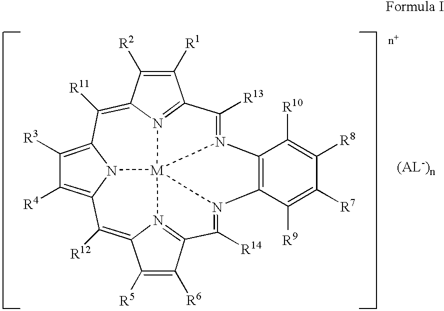
wherein:
AL is an apical ligand
M is an enriched metal, or a pure isotope thereof, having a neutron capture cross section greater than about 1,000 b;
n is an integer of 1-5;
R¹, R², R³, R⁴, R⁵, R⁶, R⁷, and R⁸ are independently chosen from the group consisting of hydrogen, halogen, hydroxyl, optionally substituted alkyl, optionally substituted alkenyl, optionally substituted alkynyl, optionally substituted aryl, optionally substituted heteroaryl, optionally substituted haloalkyl; nitro, acyl, optionally substituted alkoxy, saccharide, optionally substituted amino, carboxyl, optionally substituted carboxyalkyl, optionally substituted carboxyamide, optionally substituted carboxyamidealkyl, optionally substituted heterocycle, optionally substituted cycloalkyl, optionally substituted arylalkyl, optionally substituted heteroarylalkyl, optionally substituted heterocycloalkylalkyl, and the group —X—Y, in which X is a covalent bond or a linker and Y is a catalytic group, a chemotherapeutic agent, or a site-directing molecule; and
R⁹, R¹⁰, R¹¹, R¹², R¹³ and R¹⁴ are independently hydrogen, optionally substituted alkyl, optionally substituted aryl, optionally substituted alkoxy, optionally substituted carboxyalkyl, or optionally substituted carboxyamidealkyl;
with the proviso that the halogen is other than iodide and the haloalkyl is other than iodoalkyl.

Preferably, M is an enriched metal, more preferably selected from: enriched gadolinium, enriched cadmium, enriched europium, enriched mercury and enriched samarium. Particularly preferred are: ¹⁵⁵Gd- and/or ¹⁵⁷Gd-enriched gadolinium, ¹¹³Cd-enriched cadmium, ¹⁵¹Eu-enriched europium, ¹⁹⁹Hg-enriched mercury, and ¹⁴⁹Sm-enriched samarium.

Most preferred are those compounds of Formula I in which M is a pure isotope, preferably ¹⁵⁵Gd, ¹⁵⁷Gd, ¹¹³Cd, ¹⁵¹Eu, ¹⁹⁹Hg or ¹⁴⁹Sm, especially those compounds of Formula I where M is the pure ¹⁵⁷Gd isotope of gadolinium or ¹⁵⁷Gd-enriched gadolinium.

In a sixth aspect, the invention relates to pharmaceutical formulations, comprising a therapeutically effective amount of a compound of Formula I and at least one pharmaceutically acceptable excipient.

### DETAILED DESCRIPTION OF THE INVENTION

Neutron capture therapy (NCT) is a binary therapeutic method of treatment, particularly useful in the treatment of brain cancers. One advantage of such a two-step therapy is that each step when taken by itself has relatively little effect on normal cells; it is only when the two steps are combined that action is induced. The two steps are 1) administration of a non-toxic neutron capture agent to a patient so as to provide selective uptake and/or retention of the agent within a tumor, or plaque caused by atherosclerosis, followed by 2) irradiation of the site at which the neutron capture agent is retained with a neutron beam. The thermal (or slow) neutrons that are employed in such treatment cause little tissue damage as compared to other types of radiation commonly used in the treatment of cancer, for example ionizing radiation such as protons, gamma rays, X-rays, and fast neutrons.

For NCT to be effective, it is essential that capture of neutrons by the NCT agent results in intense short-range radiation being generated in close proximity to the cancer cell or other targeted tissue. Therefore it is critical that, upon administration, the neutron capture agent is selectively absorbed by the tumor cells or plaque as compared to normal tissue; absent such an effect, normal cells would also have NCT agent present, and thus would be destroyed upon bombardment with neutrons as well as the target tissue. It is also desirable that the neutron capture agent is retained by the tumor or plaque for a period of time sufficient to allow clearance of the NCT agent from normal tissue and vascular compartments prior to neutron exposure. Additionally, it is preferable that the atoms or isotopes responsible for capturing the neutrons have a large neutron capture cross-section, preferably greater than 1,000 b. It is also preferred that the neutron capture agent should be non-radioactive.

The present invention provides all of the above desired features. The metal-texaphyrin complexes of the invention accumulate selectively in neoplastic tissue and atheromatous plaque, and are retained for a time sufficient for unbound material to clear from normal tissue and vascular compartments. The preferred metal is gadolinium, and the preferred gadolinium isotope is ¹⁵⁷Gd, which has the highest barns radius (>254,000 b) of all of the gadolinium isotopes (this is about 66 times greater than that of ¹⁰B), and is not radioactive. Upon bombardment by neutrons, ¹⁵⁷Gd emits high LET (linear energy transfer) fission products forms of radiation, such as γ-rays and Aujer electrons, which have more energy than the products emitted by ¹⁰B (⁷Li and high energy α-particles). γ-Rays have an average energy of about 2.2 MeV, with a range of several centimeters, whereas α-particles are slower and have a shorter path length (10-14 μm). As a consequence of these properties, the compounds of the present invention are particularly well suited for use in neutron capture therapy.

Another advantage of the compounds of this invention, in addition to the role of the central metal component of the compounds in capturing slow neutrons, is their ability of the compounds themselves to augment the cytotoxic effects of radiation produced during the neutron capture process. Additionally, the compounds of the invention sensitize cells in which they are selectively retained to the effect of chemotherapeutic agents, ionizing radiation, light and/or sonic disruption, if so desired, as disclosed in the patents detailed below, whether prior, subsequent or contemporaneous with exposure to slow neutron irradiation.

### Definitions and General Parameters

As used in the present specification, the following words and phrases are generally intended to have the meanings as set forth below, except to the extent that the context in which they are used indicates otherwise.

“Natural” as employed with reference to the substituent M in Formula I or in the phrase “natural metal” means a metal having a natural abundance of isotopes (i.e., the distribution typically found for the element as it occurs in nature).

“Enriched” as employed with reference to the substituent M in Formula I or in the phrase “enriched metal” means a metal having an excess abundance of one or more isotopes such that its neutron capture cross section is greater than the neutron capture cross section of the corresponding natural metal.

Except as otherwise specified “neutron(s)” refer to “slow” or “thermal” neutrons of the type employed in neutron capture therapy.

The term “compound of Formula I” is intended to encompass the metallotexaphyrins of the invention as disclosed, coordination complexes of the compounds of Formula I, and/or the pharmaceutically acceptable salts of such compounds.

The term “effective amount” or “therapeutically effective amount” refers to that amount of a compound of Formula I that is sufficient to effect treatment, as defined below, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular compound of Formula I chosen, the dosing regimen to be followed, timing of administration, the tissue to be imaged, and the physical delivery system in which it is carried.

“Optional” or “optionally” means that the subsequently described event or circumstance may or may not occur, and that the description includes instances where said event or circumstance occurs and instances in which it does not.

The term “treatment” or “treating” means any treatment of a disease in a mammal, including:
(i) preventing the disease, that is, causing the clinical symptoms of the disease not to develop;
(ii) inhibiting the disease, that is, arresting the development of clinical symptoms; and/or
(iii) relieving the disease, that is, causing the regression of clinical symptoms.

“Epithermal neutron beam” means the type of beam wherein neutrons possess kinetic energies of between about 0.4 eV and 10 keV.

Nomenclature Definitions

The following definitions are provided for the purpose of elucidating the scope of the invention with reference to the definitions employed in describing the substituent groups associated with the compounds of the present invention. As used herein:

The term “alkyl” refers to a monoradical branched or unbranched saturated hydrocarbon chain preferably having from 1 to 20 carbon atoms, more preferably 1 to 10 carbon atoms, and even more preferably 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to
1) an alkyl group as defined above, having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl; or
2) an alkyl group as defined above that is interrupted by 1-20 atoms independently chosen from oxygen, sulfur and NR^{a}—, where R^{a} is chosen from hydrogen, or optionally substituted alkyl, cycloalkyl, alkenyl, cycloalkenyl, alkenyl, cycloalkenyl, alkynyl, aryl, heteroaryl and heterocyclic; or
3) an alkyl group as defined above that has both from 1 to 5 substituents as defined above and is also interrupted by 1-20 atoms as defined above.

One preferred alkyl substituent is hydroxy, exemplified by hydroxyalkyl groups, such as 2-hydroxyethyl, 3-hydroxypropyl, 3-hydroxybutyl, 4-hydroxybutyl, and the like; dihydroxyalkyl groups (glycols), such as 2,3-dihydroxypropyl, 3,4-dihydroxybutyl, 2,4-dihydroxybutyl, and the like; and those compounds known as polyethylene glycols, polypropylene glycols and polybutylene glycols, and the like.

The term “alkylene” refers to a diradical of a branched or unbranched saturated hydrocarbon chain, preferably having from 1 to 20 carbon atoms, preferably 1-10 carbon atoms, more preferably 1-6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “substituted alkylene” refers to:
(1) an alkylene group as defined above having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyacylamino, azido, cyano, halogen, hydroxyl, keto, thioketo, carboxyl, carboxylalkyl, thiol, thioalkoxy, substituted thioalkoxy, aryl, aryloxy, thioaryloxy, heteroaryl, heteroaryloxy, thioheteroaryloxy, heterocyclic, heterocyclooxy, thioheterocyclooxy, nitro, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic. Additionally, such substituted alkylene groups include those where two substituents on the alkylene group are fused to form one or more cycloalkyl, substituted cycloalkyl, cycloalkenyl, substituted cycloalkenyl, aryl, heterocyclic or heteroaryl groups fused to the alkylene group; or
(2) an alkylene group as defined above that is interrupted by 1-20 atoms independently chosen from oxygen, sulfur and NR^{a}—, where R^{a} is chosen from hydrogen, optionally substituted alkyl, cycloalkyl, alkenyl, cycloalkenyl, alkenyl, cycloalkenyl, alkynyl, aryl, heteroaryl and heterocyclic, or groups selected from carbonyl, carboxyester, carboxyamide and sulfonyl; or
(3) an alkylene group as defined above that has both from 1 to 5 substituents as defined above and is also interrupted by 1-20 atoms as defined above.
   Examples of substituted alkylenes are chloromethylene (—CH(Cl)—), aminoethylene (—CH(NH₂)CH₂—), 2-carboxypropylene isomers (—CH₂CH(CO₂H)CH₂—), ethoxyethyl (—CH₂CH₂O—CH₂CH₂—), ethylmethylaminoethyl (—CH₂CH₂N(CH₃)CH₂CH₂—), 1-ethoxy-2-(2-ethoxy-ethoxy)ethane (—CH₂CH₂O—CH₂CH₂—OCH₂CH₂—OCH₂CH₂—), and the like.

The term “alkaryl” refers to the groups -optionally substituted alkylene-optionally substituted aryl, where alkylene, substituted alkylene, aryl and substituted aryl are defined herein. Such alkaryl groups are exemplified by benzyl, phenethyl and the like.

The term “alkoxy” refers to the groups alkyl-O—, alkenyl-O—, cycloalkyl-O—, cycloalkenyl-O—, and alkynyl-O—, where alkyl, alkenyl, cycloalkyl, cycloalkenyl, and alkynyl are as defined herein. Preferred alkoxy groups are alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein. One preferred substituted alkoxy group is substituted alkyl-O, and includes groups such as —OCH₂CH₂OCH₃, PEG groups such as —O(CH₂CH₂O)ₓCH₃, where x is an integer of 2-20, preferably 2-10, and more preferably 2-5. Another preferred substituted alkoxy group is —O—CH₂—(CH₂)_{y}—OH, where y is an integer of 1-10, preferably 1-4.

The term “alkylalkoxy” refers to the groups -alkylene-O-alkyl, alkylene-O-substituted alkyl, substituted alkylene-O-alkyl and substituted alkylene-O-substituted alkyl wherein alkyl, substituted alkyl, alkylene and substituted alkylene are as defined herein. Preferred alkylalkoxy groups are alkylene-O-alkyl and include, by way of example, methylenemethoxy (—CH₂OCH₃), ethylenemethoxy (—CH₂CH₂OCH₃), n-propylene-iso-propoxy (—CH₂CH₂CH₂OCH(CH₃)₂), methylene-t-butoxy (—CH₂—O—C(CH₃)₃) and the like.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein. Preferred alkylthioalkoxy groups are alkylene-S-alkyl and include, by way of example, methylenethiomethoxy (—CH₂SCH₃), ethylenethiomethoxy (—CH₂CH₂SCH₃), n-propylene-iso-thiopropoxy (—CH₂CH₂CH₂SCH(CH₃)₂), methylene-t-thiobutoxy (—CH₂SC(CH₃)₃) and the like.

The term “alkenyl” refers to a monoradical of a branched or unbranched unsaturated hydrocarbon group preferably having from 2 to 20 carbon atoms, more preferably 2 to 10 carbon atoms and even more preferably 2 to 6 carbon atoms and having at least 1 and preferably from 1-6 sites of vinyl unsaturation. Preferred alkenyl groups include ethenyl (—CH═CH₂), n-propenyl (—CH₂CH═CH₂), iso-propenyl (—C(CH₃)═CH₂), and the like.

The term “substituted alkenyl” refers to an alkenyl group as defined above having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkenylene” refers to a diradical of a branched or unbranched unsaturated hydrocarbon group preferably having from 2 to 20 carbon atoms, more preferably 2 to 10 carbon atoms and even more preferably 2 to 6 carbon atoms and having at least 1 and preferably from 1-6 sites of vinyl unsaturation. This term is exemplified by groups such as ethenylene (—CH═CH—), the propenylene isomers (e.g., —CH₂CH═CH— and —C(CH₃)═CH—) and the like.

The term “substituted alkenylene” refers to an alkenylene group as defined above having from 1 to 5 substituents, and preferably from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Additionally, such substituted alkenylene groups include those where 2 substituents on the alkenylene group are fused to form one or more cycloalkyl, substituted cycloalkyl, cycloalkenyl, substituted cycloalkenyl, aryl, heterocyclic or heteroaryl groups fused to the alkenylene group.

The term “alkynyl” refers to a monoradical of an unsaturated hydrocarbon, preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynyl groups include ethynyl, (—C≡CH), propargyl (—C≡CCH₃), and the like.

The term “substituted alkynyl” refers to an alkynyl group as defined above having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkynylene” refers to a diradical of an unsaturated hydrocarbon preferably having from 2 to 20 carbon atoms, more preferably 2 to 10 carbon atoms and even more preferably 2 to 6 carbon atoms and having at least 1 and preferably from 1-6 sites of acetylene (triple bond) unsaturation. Preferred alkynylene groups include ethynylene (—C≡C—), propargylene (—CH₂—C≡C—) and the like.

The term “substituted alkynylene” refers to an alkynylene group as defined above having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “acyl” refers to the groups HC(O)—, alkyl-C(O)—, substituted alkyl-C(O)—, cycloalkyl-C(O)—, substituted cycloalkyl-C(O)—, cycloalkenyl-C(O)—, substituted cycloalkenyl-C(O)—, aryl-C(O)—, heteroaryl-C(O)— and heterocyclic-C(O)— where alkyl, substituted alkyl, cycloalkyl, substituted cycloalkyl, cycloalkenyl, substituted cycloalkenyl, aryl, heteroaryl and heterocyclic are as defined herein.

The term “acylamino” or “aminocarbonyl” refers to the group —C(O)NRR where each R is independently hydrogen, alkyl, substituted alkyl, aryl, heteroaryl, heterocyclic or where both R groups are joined to form a heterocyclic group (e.g., morpholino) wherein alkyl, substituted alkyl, aryl, heteroaryl and heterocyclic are as defined herein.

The term “aminoacyl” refers to the group —NRC(O)R where each R is independently hydrogen, alkyl, substituted alkyl, aryl, heteroaryl, or heterocyclic wherein alkyl, substituted alkyl, aryl, heteroaryl and heterocyclic are as defined herein.

The term “aminoacyloxy” or “alkoxycarbonylamino” refers to the group —NRC(O)OR where each R is independently hydrogen, alkyl, substituted alkyl, aryl, heteroaryl, or heterocyclic wherein alkyl, substituted alkyl, aryl, heteroaryl and heterocyclic are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclic-C(O)O— wherein alkyl, substituted alkyl, cycloalkyl, substituted cycloalkyl, aryl, heteroaryl, and heterocyclic are as defined herein.

The term “aryl” refers to an unsaturated aromatic carbocyclic group of from 6 to 20 carbon atoms having a single ring (e.g., phenyl) or multiple condensed (fused) rings (e.g., naphthyl or anthryl). Preferred aryls include phenyl, naphthyl and the like.

Unless otherwise constrained by the definition for the aryl substituent, such aryl groups can optionally be substituted with from 1 to 5 substituents, preferably 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Preferred aryl substituents include alkyl, alkoxy, halo, cyano, nitro, trihalomethyl, and thioalkoxy.

The term “aryloxy” refers to the group aryl-O— wherein the aryl group is as defined above including optionally substituted aryl groups as also defined above.

The term “arylene” refers to the diradical derived from aryl (including substituted aryl) as defined above and is exemplified by 1,2-phenylene, 1,3-phenylene, 1,4-phenylene, 1,2-naphthylene and the like.

The term “amino” refers to the group —NH₂.

The term “substituted amino refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic provided that both R's are not hydrogen.

The term “carboxyalkyl” or “alkoxycarbonyl” refers to the groups “—C(O)O-alkyl”, “—C(O)O-substituted alkyl”, “—C(O)O-cycloalkyl”, “—C(O)O-substituted cycloalkyl”, “—C(O)O-alkenyl”, “—C(O)O-substituted alkenyl”, “—C(O)O-alkynyl” and “—C(O)O-substituted alkynyl” where alkyl, substituted alkyl, cycloalkyl, substituted cycloalkyl, alkenyl, substituted alkenyl, alkynyl and substituted alkynyl are as defined herein.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “cycloalkylene” refers to the diradical derived from cycloalkyl as defined above and is exemplified by 1,1-cyclopropylene, 1,2-cyclobutylene, 1,4-cyclohexylene and the like.

The term “substituted cycloalkyl” refers to cycloalkyl groups having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted cycloalkylene” refers to the diradical derived from substituted cycloalkyl as defined above.

The term “cycloalkenyl” refers to cyclic alkenyl groups of from 4 to 20 carbon atoms having a single cyclic ring and at least one point of internal unsaturation. Examples of suitable cycloalkenyl groups include, for instance, cyclobut-2-enyl, cyclopent-3-enyl, cyclooct-3-enyl and the like.

The term “cycloalkenylene” refers to the diradical derived from cycloalkenyl as defined above and is exemplified by 1,2-cyclobut-1-enylene, 1,4-cyclohex-2-enylene and the like.

The term “substituted cycloalkenyl” refers to cycloalkenyl groups having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted cycloalkenylene” refers to the diradical derived from substituted cycloalkenyl as defined above.

The term “halo” or “halogen” refers to fluoro, chloro, bromo and iodo.

The term “heteroaryl” refers to an aromatic group comprising 1 to 15 carbon atoms and 1 to 4 heteroatoms selected from oxygen, nitrogen and sulfur within at least one ring (if there is more than one ring).

Unless otherwise constrained by the definition for the heteroaryl substituent, such heteroaryl groups can be optionally substituted with 1 to 5 substituents, preferably 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Preferred aryl substituents include alkyl, alkoxy, halo, cyano, nitro, trihalomethyl, and thioalkoxy. Such heteroaryl groups can have a single ring (e.g., pyridyl or furyl) or multiple condensed rings (e.g., indolizinyl or benzothienyl). Preferred heteroaryls include pyridyl, pyrrolyl and furyl.

The term “heteroaryloxy” refers to the group heteroaryl-O—.

The term “heteroarylene” refers to the diradical group derived from heteroaryl (including substituted heteroaryl), as defined above, and is exemplified by the groups 2,6-pyridylene, 2,4-pyridiylene, 1,2-quinolinylene, 1,8-quinolinylene, 1,4-benzofuranylene, 2,5-pyridnylene, 2,5-indolenyl and the like.

The term “heterocycle” or “heterocyclic” refers to a monoradical saturated or unsaturated group having a single ring or multiple condensed rings, having from 1 to 40 carbon atoms and from 1 to 10 hetero atoms, preferably 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Such heterocyclic groups can have a single ring or multiple condensed rings. Preferred heterocyclics include morpholino, piperidinyl, and the like.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

The term “heterocyclooxy” refers to the group heterocyclic-O—.

The term “thioheterocyclooxy” refers to the group heterocyclic-S—.

The term “heterocyclene” refers to the diradical group formed from a heterocycle, as defined herein, and is exemplified by the groups 2,6-morpholino, 2,5-morpholino and the like.

The term “oxyacylamino” or “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

The term “spiro-attached cycloalkyl group” refers to a cycloalkyl group attached to another ring via one carbon atom common to both rings.

The term “thiol” refers to the group —SH.

The term “thioalkoxy” refers to the group —S-alkyl.

The term “substituted thioalkoxy” refers to the group —S-substituted alkyl.

The term “thioaryloxy” refers to the group aryl-S— wherein the aryl group is as defined above including optionally substituted aryl groups also defined above.

The term “thioheteroaryloxy” refers to the group heteroaryl-S— wherein the heteroaryl group is as defined above including optionally substituted aryl groups as also defined above.

As to any of the above groups that contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

The term “carboxyamides” include primary carboxyamides (CONH₂), secondary carboxyamides (CONHR′) and tertiary carboxyamides (CONR′R″), where R′ and R″ are the same or different substituent groups chosen from alkyl, alkenyl, alkynyl, alkoxy, aryl, a heterocyclic group, a functional group as defined herein, and the like, which themselves may be substituted or unsubstituted.

“Carboxyamidealkyl” means a carboxyamide as defined above attached to an optionally substituted alkylene group as defined above.

The term “saccharide” includes oxidized, reduced or substituted saccharides, including hexoses such as D-glucose, D-mannose or D-galactose; pentoses such as D-ribose or D-arabinose; ketoses such as D-ribulose or D-fructose; disaccharides such as sucrose, lactose, or maltose; derivatives such as acetals, amines, and phosphorylated sugars; oligosaccharides; as well as open chain forms of sugars, and the like. Examples of amine-derivatized sugars are galactosamine, glucosamine, and sialic acid.

The term “site-directing molecule” refers to a molecule having an affinity for a biological receptor or for a nucleic acid sequence. Exemplary site-directing molecules useful herein include, but are not limited to, polydeoxyribonucleotides, oligodeoxyribonucleotides, polyribonucleotide analogs, oligoribonucleotide analogs, polyamides including peptides having affinity for a biological receptor and proteins such as antibodies, steroids and steroid derivatives, hormones such as estradiol or histamine, hormone mimics such as morphine, and further macrocycles such as sapphyrins and rubyrins. The oligonucleotides may be derivatized at the bases, the sugars, the ends of the chains, or at the phosphate groups of the backbone to promote in vivo stability. Modifications of the phosphate groups are preferred in one embodiment since phosphate linkages are sensitive to nuclease activity. Presently preferred derivatives are the methylphosphonates, phosphotriesters, phosphorothioates, and phosphoramidates. Additionally, the phosphate linkages may be completely substituted with non-phosphate linkages such as amide linkages. Appendages to the ends of the oligonucleotide chains also provide exonuclease resistance. Sugar modifications may include groups, such as halo, alkyl, alkenyl or alkoxy groups, attached to an oxygen of a ribose moiety in a ribonucleotide. In a preferred embodiment, the group will be attached to the 2′ oxygen of the ribose. In particular, halogen moieties such as fluoro may be used. The alkoxy group may be methoxy, ethoxy or propoxy. The alkenyl group is preferably allyl. The alkyl group is preferably a methyl group and the methyl group is attached to the 2′ oxygen of the ribose. Other alkyl groups may be ethyl or propyl. It is understood that the terms “nucleotide”, “polynucleotide” and “oligonucleotide”, as used herein and in the appended claims, refer to both naturally-occurring and synthetic nucleotides, poly- and oligonucleotides and to analogs and derivatives thereof such as methylphosphonates, phosphotriesters, phosphorothioates, phosphoramidates and the like. Deoxyribonucleotides, deoxyribonucleotide analogs and ribonucleotide analogs are contemplated as site-directing molecules in the present invention. The term “texaphyrin-oligonucleotide conjugate” means that an oligonucleotide is attached to the texaphyrin in a 5′ or a 3′ linkage, or in both types of linkages to allow the texaphyrin to be an internal residue in the conjugate. It can also refer to a texaphyrin that is linked to an internal base of the oligonucleotide. The oligonucleotide or other site-directing molecule may be attached either directly to the texaphyrin or to the texaphyrin via a linker or a couple of variable length.

The term “catalytic group” means a chemical functional group that assists catalysis by acting as a general acid, Brønsted acid, general base, Brønsted base, nucleophile, or any other means by which the activation barrier to reaction is lowered or the ground state energy of the substrate is increased. Exemplary catalytic groups contemplated include, but are not limited to, imidazole; guanidine; substituted saccharides such as D-glucosamine, D-mannosamine, D-galactosamine, D-glucamine and the like; amino acids such as L-histidine and L-arginine; derivatives of amino acids such as histamine; polymers of amino acids such as poly-L-lysine, (LysAla), (LysLeuAla)ₙ where n is from 1-30 or preferably 1-10 or more preferably 2-7 and the like; derivatives thereof; and metallotexaphyrin complexes.

A “chemotherapeutic agent” may be, but is not limited to, one of the following: an alkylating agent such as a nitrogen mustard, an ethyleneimine or a methylmelamine, an alkyl sulfonate, a nitrosourea, or a triazene; an antimetabolite such as a folic acid analog, a pyrimidine analog, or a purine analog; a natural product such as a vinca alkaloid, an epipodophyllotoxin, an antibiotic, an enzyme, taxane, or a biological response modifier; miscellaneous agents such as a platinum coordination complex such as cisplatin, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, or a gonadotropin-releasing hormone analog. Chemotherapeutic agents are used in the treatment of cancer and other neoplastic tissue. Preferably, the chemotherapeutic agent is a nitrogen mustard, an epipodophyllotoxin, an antibiotic, or a platinum coordination complex. A more preferred chemotherapeutic agent is bleomycin, doxorubicin, taxol, taxotere, etoposide, 4-OH cyclophosphamide, cisplatin, or platinum coordination complexes analogous to cisplatin. A presently preferred chemotherapeutic agent is doxorubicin, taxol, taxotere, cisplatin, or Pt complexes analogous to cisplatin. Various chemotherapeutic agents, their target diseases, and treatment protocols are presented in, for example, Goodman and Gilman's The Pharmacological Basis of Therapeutics, Eighth Ed., Pergamon Press, Inc., 1990; and Remington: The Science and Practice of Pharmacy, Mack Publishing Co., Easton, Pa., 1995; both of which are incorporated by reference herein.

A site directing molecule, or a group having or catalytic or chemotherapeutic activity, identified above by the symbol Y, may be covalently coupled to any position on a macrocycle (for example a texaphyrin or a sapphyrin) by a covalent bond or by a linker (identified above by the symbol X). The term “linker” as used herein means a group that covalently connects Y to a macrocycle, and may be, for example, alkylene, alkenylene, alkynylene, arylene, ethers, PEG moieties, and the like, all of which may be optionally substituted. Examples of reactions to form a covalent link include reaction between an amine (on either the molecule Y or X) with a carboxylic acid (on the corresponding X or Y) to form an amide link. Similar reactions well known in the art are described in standard organic chemistry texts such as J. March, “Advanced Organic Chemistry”, 4^{th} Edition, Wiley-Interscience, New York (1992).

The term “apical ligand” means an anionic or neutral species that binds to the core metal via coordinative or de-localized electrostatic bonds, or both. In general, any electron-rich species may act as an apical ligand, for example carboxylates and phosphates, and species containing these functional groups. Preferred apical ligands include lipoproteins, amino acids, biomolecules, carboxylates of sugar derivatives, such as gluconic acid or glucoronic acid, cholesterol derivatives such as cholic acid and deoxycholic acid, PEG acids, organophosphates, such as methylphosphonic acid and phenylphosphonic acid, and phosphoric acid or other inorganic acids, and the like. Preferred are “carboxylic acid derivatives”, which term refers to compounds of the formula R—CO₂H, in which R is optionally substituted alkyl, optionally substituted alkenyl, optionally substituted alkynyl, or optionally substituted aryl, as defined above. More preferred are those carboxylic acid derivatives where R is alkyl, for example acids of 1-20 carbon atoms, such as formic acid, acetic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, and the like. Also preferred are those carboxylic acid derivatives where R is aryl, in particular where R is optionally substituted phenyl, for example benzoic acid, salicylic acid, 3-fluorobenzoic acid, 4-aminobenzoic acid, cinnamic acid, mandelic acid, p-toluene-sulfonic acid, and the like.

“Texaphyrin” means an aromatic pentadentate macrocyclic expanded porphyrins, also described as an aromatic benzannulene containing both 18π- and 22π-electron delocalization pathways. For the purpose of this specification, the term texaphyrin includes both the metallated and unmetallated compounds. Texaphyrins and water-soluble texaphyrins, method of preparation and various uses have been described in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, and 5,714,328; PCT publications WO 90/10633, 94/29316, 95/10307, 95/21845, 96/09315, 96/40253, 96/38461, 97/26915, 97/35617, 97/46262, and 98/07733; allowed U.S. patent application Ser. Nos. 08/458,347, 08/591,318, and 08/914,272; and pending U.S. patent application Ser. Nos. 08/763,451, 08/903,099, 08/946,435, 08/975,090, 08/975,522, 08/988,336, and 08/975,526; each previously incorporated herein by reference.

The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The compounds of Formula I are capable of forming salts, and “coordination complexes”, with molecules such as pyridine, benzimidazole, water, and methanol.

Compounds of the Invention

The compounds of the invention are metallotexaphyrins, in which the central component is a metal, an enriched metal, or a pure isotopr thereof, having a neutron capture cross-section greater than about 1,000 b, and are represented by Formula I: 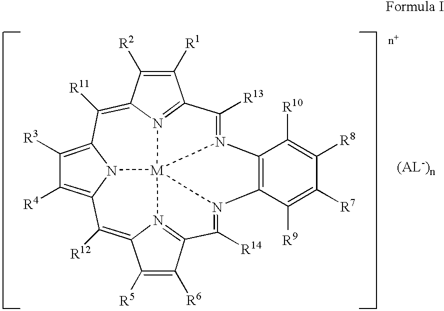
wherein:
AL is an apical ligand;
M is a metal, an enriched metal, or a pure isotope thereof, having a neutron capture cross section greater than about 1,000 b;
n is an integer of 1-5;
R¹, R², R³, R⁴, R⁵, R⁶, R⁷, and R⁸ are independently chosen from the group consisting of hydrogen, halogen, hydroxyl, optionally substituted alkyl, optionally substituted alkenyl, optionally substituted alkynyl, optionally substituted aryl, optionally substituted heteroaryl, optionally substituted haloalkyl; nitro, acyl, optionally substituted alkoxy, saccharide, optionally substituted amino, carboxyl, optionally substituted carboxyalkyl, optionally substituted carboxyamide, optionally substituted carboxyamidealkyl, optionally substituted heterocycle, optionally substituted cycloalkyl, optionally substituted arylalkyl, optionally substituted heteroarylalkyl, optionally substituted heterocycloalkylalkyl, and the group —X—Y, in which X is a covalent bond or a linker and Y is a catalytic group, a chemotherapeutic agent, or a site-directing molecule; and
R⁹, R¹⁰, R¹¹, R¹², R¹³ and R¹⁴ are independently hydrogen, optionally substituted alkyl, optionally substituted aryl, optionally substituted alkoxy, optionally substituted carboxyalkyl, or optionally substituted carboxyamidealkyl;

Examples of metals suitable for the compounds of the present invention, and the preferred isotope of those metals, are shown in the table below:

| METAL | ISOTOPE | BARNS RADIUS | |
|---|---|---|---|
| Gadolinium | ¹⁵⁵ Gd | 60,948 | b |
| Gadolinium | ¹⁵⁷ Gd | >254,000 | b |
| Cadmium | ¹¹³ Cd | 20,673 | b |
| Europium | ¹⁵¹ Eu | 9,201 | b |
| Mercury | ¹⁹⁹ Hg | 2,100 | b |
| Samarium | ¹⁴⁹ Sm | 40,326 | b |

The natural metal “gadolinium” (Gd) is a rare earth element having seven naturally occurring isotopes, the abundances and barns radii of which are shown in the table below:

| ISOTOPE | ABUNDANCE | BARNS RADIUS | |
|---|---|---|---|
| ¹⁵² Gd | 0.20% | 900 | b |
| ¹⁵⁴ Gd | 2.15% | 0.06 | b |
| ¹⁵⁵ Gd | 14.73% | 61,000 | b |
| ¹⁵⁶ Gd | 20.47% | 2.0 | b |
| ¹⁵⁷ Gd | 15.68% | >254,000 | b |
| ¹⁵⁸ Gd | 24.87% | 2.3 | b |
| ¹⁶⁰ Gd | 21.90% | 1.5 | b |
| Natural Gd | 100% | 48,800 | b |

In considering the above table, it can be seen that the barns radius of natural gadolinium is a weighted average of the barns radii of its naturally occurring isotopes. This weighted average barns radius can be increased by employing mixtures of selected gadolinium isotopes, constituting an enriched gadolinium. For example, ¹⁵²Gd is itself radioactive and has a comparatively small barns radius, so it would not be preferred as an isotope for enriched gadolinium. ¹⁵⁵Gd and ¹⁵⁷Gd both absorb thermal and epithermal neutrons, and have barns radii greater than normal gadolinium. Thus, as used in the compounds and methods of the present invention, enriched gadolinium comprises, e.g., more than about 17% ¹⁵⁷Gd. Another enriched gadolinium comprises more than about 18% ¹⁵⁵Gd. Preferred enriched gadolinium metals can comprise up to and including 100% of an enriching isotope, more preferably (and for practical purposes) about 20% to 95%, and most preferably at least 50% of an enriching isotope. The same analysis and criteria pertain to the other enriched metals employed in the present invention.

Another advantage for ¹⁵⁷Gd as compared to ¹⁰B is that upon impact with thermal neutrons, ¹⁵⁷Gd emits forms of radiation different from those seen for ¹⁰B. Although not wishing to be bound by theory, it is believed that upon bombardment by neutrons, ¹⁵⁷Gd emits high LET (linear energy transfer) forms of radiation, such as γ-rays and Aujer electrons, which have more energy than the products emitted by ¹⁰B (⁷Li and α-particles), and thus have more potential for causing damage to cancer cells. γ-Rays have an average energy of about 2.2 MeV, with a range of several centimeters, whereas α-particles are slower and have a shorter path length (10-14 μm).

Preferred Compounds

Preferred are the compounds of Formula I in which M is a divalent or trivalent metal, R¹ is hydroxyalkyl (in which alkyl preferably has 1-10 carbon atoms), R², R³ and R⁴ are alkyl (preferably of 1-6 carbon atoms), R⁷ and R⁸ are substituted alkoxy (in which alkoxy preferably has 1-20 carbon atoms), and n is 1-4. R⁵, R⁶, R⁹, R¹⁰, R¹¹ and R¹² are hydrogen or alkyl of 1-6 carbon atoms.

More preferred are the compounds of Formula I in which R² and R⁵ are both —CH₂(CH₂)₂OH, R³ and R⁴ are both —CH₂CH₃, R¹ and R⁶ are both —CH₃, R⁷ and R⁸ are both —OCH₂CH₂OCH₂OR, where R is hydrogen or —CH₃, and R⁹-R¹⁴ are hydrogen, and the apical ligand AL is acetate. Most preferred are the compounds in which R⁷ and R⁸ are both —OCH₂CH₂OCH₂OCH₃. The preferred metal is gadolinium, which may be used as the naturally occurring element, having 15.68% of the ¹⁵⁷gadolinium isotope present. More preferred is gadolinium enriched in the ¹⁵⁷gadolinium isotope to a degree approximating 50% or greater than that which normally occurs in the naturally occurring element. Most preferred is the pure ¹⁵⁷gadolinium isotope.

The preferred method of neutron irradiation is by a linear accelerator, preferably performed about 2-24 hours after administration of the gadolinium texaphyrin. The preferred amount of irradiation by a linear accelerator is between the range of 1-5×10¹² neutrons per cm².

Nomenclature

The compounds of Formula IA may be named and numbered in several different ways, (e.g. depending on the origination of the numbering). One example of numbering is shown described below with reference to Formula I.

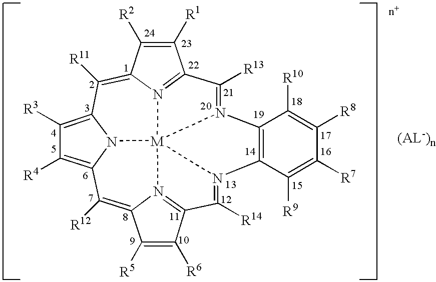

For example, the specific gadolinium texaphyrin molecule (CAS Registry No. 156436-89-4) is shown below: 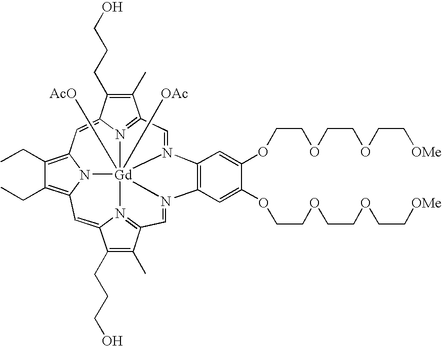
This compound can also be named in a variety of ways. Examples of alternative names for this compound are:
The gadolinium(III) complex of:
4,5-diethyl-10,23-dimethyl-9,24-bis(3-hydroxypropyl)-16,17-bis[2-[2-(2 methoxyethoxy)ethoxy]ethoxy]pentaazapentacyclo[20.2.1.1^{3,6}.1^{8,11}.0^{14,19}]heptacos a-1,3,5,7,9,11(27),12,14,16,18,20,22(25),23-tridecaene bis acetate.
   Alternatively, the Chemical Abstracts name is bis(acetato-O)[9,10-diethyl-20,21-bis[2-[2-(2-methoxyethoxy)ethoxy]ethoxy]-4,15-dimethyl-8,11-imino-3,6:16,13-dinitrilo-1,18-benzodiazacycloeicosine-5,14-dipropanolato-N1,N18,N23,N24,N25]gadolinium. The compound is also called by the trivial names Gadolinium Texaphyrin, Gd texaphyrin and Gd-Tex, or the generic name or USAN motaxafin gadolinium, and has the internal designation PCI-0120 and the trademark XCYTRIN™.

Another example of a compound of the invention is:

The ¹⁵⁷Gd complex of 4,5-diethyl-10,23-dimethyl-9,24-bis(3-hydroxy propyl)-16,17-bis[2-[2-(2-methoxyethoxy)ethoxy]ethoxy]pentaazapentacyclo[20.2.1.1^{3,6}.1^{8,11}.0^{14,19}]heptacos a-1,3,5,7,9,11(27),12,14,16,18,20,22(25),23-tridecaene bis acetate.

### Synthesis of the Compounds of Formula I

### Synthetic Reaction Parameters

The terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary, the solvents used in the reactions of the present invention are inert organic solvents.

The term “q.s.” means adding a quantity sufficient to achieve a stated function, e.g., to bring a solution to the desired volume (i.e., 100%).

Unless specified to the contrary, the reactions described herein take place at atmospheric pressure within a temperature range from 5° C. to 100° C. (preferably from 10° C. to 50° C.; most preferably at “room” or “ambient” temperature, e.g., 20° C.). Further, unless otherwise specified, the reaction times and conditions are intended to be approximate, e.g., taking place at about atmospheric pressure within a temperature range of about 5° C. to about 100° C. (preferably from about 10° C. to about 50° C.; most preferably about 20° C.) over a period of about 1 to about 10 hours (preferably about 5 hours). Parameters given in the Examples are intended to be specific, not approximate.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. Other equivalent separation or isolation procedures can, of course, also be used.

Syntheses

The compounds of Formula I can be prepared by following the procedures described in U.S. Pat. Nos. 5,569,759 and 5,801,229 incorporated herein by reference, replacing gadolinium with an appropriate metal, for example Ca⁺², Mn⁺², Co⁺², Ni⁺², Zn⁺², Cd⁺², Hg⁺², Sm⁺², UO⁺², Mn⁺³, Co⁺³, Ni⁺³, Fe⁺³, Ho⁺³, Ce⁺³, Y⁺³, In⁺³, Pr⁺³, Nd⁺³, Sm⁺³, Eu⁺³ Gd⁺³ Tb⁺³, Dy⁺³, Er⁺³, Tm⁺³, Yb⁺³, Lu⁺³, La⁺³ or U⁺³. Preferably, the metal is enriched, more preferably selected from: enriched gadolinium, enriched cadmium, enriched europium, enriched mercury and enriched samarium. Particularly preferred are: ¹⁵⁵Gd- and/or ¹⁵⁷Gd-enriched gadolinium, ¹¹³Cd-enriched cadmium, ¹⁵¹Eu-enriched europium, ¹⁹⁹Hg-enriched mercury, and ¹⁴⁹Sm-enriched samarium.

For example, to prepare the compounds of Formula I enriched in ¹⁵⁷gadolinium, the procedures described in U.S. Pat. Nos. 5,569,759 and 5,801,229 were followed, substituting gadolinium by enriched ¹⁵⁷gadolinium or pure ¹⁵⁷gadolinium, to provide the ¹⁵⁷Gd complex of 4,5-diethyl-10,23-dimethyl-9,24-bis(3-hydroxy propyl)-16,17-bis[2-[2-(2-methoxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis acetate. (enriched ¹⁵⁷gadolinium can be purchased from, for example, Oak Ridge National Laboratory, Oak Ridge Tenn.).

Compounds of Formula I enriched in ¹⁵⁷gadolinium may be prepared in any degree of enrichment desired by mixing naturally occurring Gd-Tex with isotopically pure or enriched ¹⁵⁷gadolinium-Tex in the appropriate proportions, as illustrated below in Reaction Scheme 1.

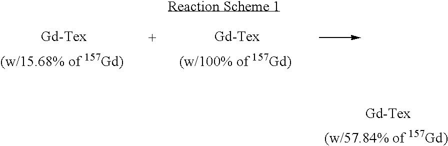

Similarly, the following compounds of Formula I are prepared as metal complexes, as enriched metal complexes, or as pure metal isotope complexes:
¹⁵⁷Gd complex of 4,5-diethyl-10,23-dimethyl-9,24-bis(3-hydroxypropyl)-16,17-bis[2-[2-(2-methoxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis gluconate;
¹⁵⁷Gd complex of 4,5-diethyl-10,23-dimethyl-9,24-bis(3-hydroxypropyl)-16,17-bis[2-[2-(2-methoxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis glucuronate;
¹⁵⁷Gd complex of 4,5-diethyl-10,23-dimethyl-9,24-bis(3-hydroxypropyl)-16,17-bis[2-[2-(2-methoxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis methylvalerate;
¹⁵⁵Gd complex of 4,5-diethyl-10,23-dimethyl-9,24-bis(3-hydroxypropyl)-16,17-bis[2-[2-(2-methoxyethoxy)ethoxy]ethoxy]pentaazapentacyclo[20.2.1.1^{3,6}.1^{8,11}.0^{14,19}]heptacos a-1,3,5,7,9,11(27),12,14,16,18,20,22(25),23-tridecaene bis acetate;
¹¹³Cd complex of 4,5-diethyl-10,23-dimethyl-9,24-bis(3-hydroxypropyl)-16,17-bis[2-[2-(2-methoxyethoxy)ethoxy]ethoxy]pentaazapentacyclo[20.2.1.1^{3,6}.1^{8,11}.0^{14,19}]heptacos a-1,3,5,7,9,11(27),12,14,16,18,20,22(25),23-tridecaene bis acetate;
¹⁵¹Eu complex of 4,5-diethyl-10,23-dimethyl-9,24-bis(3-hydroxypropyl)-16,17-bis[2-[2-(2-methoxyethoxy)ethoxy]ethoxy]pentaazapentacyclo[20.2.1.1^{3,6}.1^{8,11}.0^{14,19}]heptacos a-1,3,5,7,9,11(27),12,14,16,18,20,22(25),23-tridecaene bis acetate;
¹⁹⁹Hg complex of 4,5-diethyl-10,23-dimethyl-9,24-bis(3-hydroxypropyl)-16,17-bis[2-[2-(2-methoxyethoxy)ethoxy]ethoxy]pentaazapentacyclo[20.2.1.1^{3,6}.1^{8,11}.0^{14,19}]heptacos a-1,3,5,7,9,11(27),12,14,16,18,20,22(25),23-tridecaene bis acetate;
¹⁴⁹Sm complex of 4,5-diethyl-10,23-dimethyl-9,24-bis(3-hydroxypropyl)-16,17-bis[2-[2-(2-methoxyethoxy)ethoxy]ethoxy]pentaazapentacyclo[20.2.1.1^{3,6}.1^{8,11}.0^{14,19}]heptacos a-1,3,5,7,9,11(27),12,14,16,18,20,22(25),23-tridecaene bis acetate;
¹⁵⁷Gd complex of 4,5-dimethyl-10,23-diethyl-9,24-bis(2-hydroxyethyl)-16,17-bis[2-[2-(2-hydroxyethoxy)ethoxy]ethoxy]pentaazapentacyclo-[20.2.1.1^{3,6}1^{8,11}.0^{14,19}]heptacosa-1,3,5,7,9,11(27),12,14,16,18,20,22(25),23-tridecaene bis acetate;
¹⁵⁷Gd complex of 4,5-dimethyl-10,23-diethyl-9,24-bis(4-hydroxybutyl)-16,17-bis[2-[2-(2-methoxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene gluconate;
¹⁵⁷Gd complex of 4,5-difluoro-10,23-dimethyl-9,24-bis(2-hydroxyethyl)-16,17-bis[2-[2-(2-hydroxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis gluconate;
¹⁵⁷Gd complex of 4-phenyl-5-ethyl-10,23-diethyl-9,24-bis(2-hydroxyethyl)-16,17-bis[2-[2-(2-ethoxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis gluconate;
¹⁵⁷Gd complex of 4,5-dimethyl-10,23-diethyl-9,24-bis(2,3-dihydroxypropyl)-16,17-bis[2-[2-(2-methoxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis gluconate;
¹⁵⁷Gd complex of 4,5-dihydroxy-10,23-diethyl-9,24-bis(2-hydroxyethyl)-16,17-bis[2-[2-(2-hydroxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis glucuronate; and
¹⁵⁷Gd complex of 4,5-bis(dimethylamino)-10,23-diethyl-9,24-bis(2-hydroxyethyl)-16,17-bis[2-[2-(2-ethoxyethoxy)ethoxy]ethoxy]pentaazapentacyclo-[20.2.1.1^{3,6}.1^{8,11}.0^{14,19}]heptacosa-1,3,5,7,9,11(27),12,14,16,18,20,22(25),23-tridecaene bis gluconate.
   Neutron Capture Therapy

In the present invention, neutron capture therapy is provided to a mammal in need thereof by the co-administration of effective amounts of a metal texaphyrin complex, followed by neutron irradiation. It has surprisingly been discovered that unlike Gd-DTPA and Gd-DOTA, which have to be administered in large quantities to be potentially effective, and unlike the gadolinium porphyrins, which are not stable, the metallotexaphyrin complexes of Formula I, in particular the gadolinium-texaphyrin complexes, especially those enriched in ¹³⁷gadolinium, are suitable neutron capture agents, and are also useful as MRI contrast agents. Neutron capture therapy offers certain advantages over existing ionizing radiation and photodynamic therapies. For example, neutrons have a greater penetrative index through tissue than light, facilitating greater access to non-invasive therapy, and can also be focused more effectively as compared to photon radiation.

The precise mechanism of action of the metals and metal isotopes of the invention in neutron capture therapy remains to be definitively established. While not wanting to be bound by any particular theory, it is thought that the capture of a thermal neutron by Gd (using ¹⁵⁷gadolinium as an example), results in the production of gamma rays, Auger and conversion electrons, in the following equation:
¹⁵⁷Gd+n→→[¹⁵⁸Gd]+e⁻+δ-rays (2.2 MeV)
Electrons emitted by ¹⁵⁸Gd are high LET particles with tissue pathlengths of 1 μm to 2.5 cm and are effective for tumor therapy in NCT (?CITE?). The majority of gamma rays produced in the n+157Gd capture have an average energy of 2.2 MeV, which has been shown to be sufficient to induce DNA double-strand breaks. Thus, because ¹⁵⁷Gd-Tex localizes in the tumor cell, NCT can be used to cause selective cell death.

### Utility, Testing and Administration

General Utility

Neutron capture therapy employing the texaphyrins of the present invention is effective in the treatment of all of those particular conditions known to respond to neutron capture therapy. Included are diseases characterized by neoplastic tissue, for example cancers of the brain, mammaries, lung, liver, pancreas, colon, bladder, prostate, cervix and ovary, and sarcomas, lymphomas, leukemias, carcinomas and melanomas. Other diseases usefully treated by the compounds of the present invention in neutron capture therapy are those related to atherosclerosis, and in particular the removal of plaque.

Testing

Activity testing is conducted as described in the following references and by modifications thereof. The in vivo and in vitro activity of ¹⁵⁷Gadolinium as a neutron capture agent has been described in, for example, Brugger and Shih, 165(2-3); Evaluation of Gadolinium-157 as a neutron capture therapy agent, Strahlenther Onkol. (1989); Gadolinium Neutron Capture Therapy, Hoffman et al, Invest. Radiol. 1999; 34: 126-133.

In vitro activity for neutron capture therapy is determined, e.g., by measuring the effect of low-level, thermal neutron irradiation on the V79 brain tumor cell line in culture, and measuring resultant cytotoxicity, for example as described by Fairchild et al., Cancer Research 50. After the V79 cells are exposed to a neutron capture agent, they are irradiated with a neutron beam at a fluence rate of 2.8×10¹¹ n/cm² min. The cells are plated for undisturbed colony growth for 5-6 days, after which the remaining cell population is assessed.

In vivo activity for neutron capture therapy is determined, e.g., by post-mortem histological examination of brain tumor tissue by hematoxylin and eosin, as described in Saris et al., Cancer Research, vol. 52 (1992). The antitumor effects of neutron capture therapy are evaluated by implanting Glioma 261 tumor fragments into the brain of a mouse. Approximately 3 hours after administration of a neutron capture agent to be tested, the head is irradiated a with 1.2×10¹² neutron beam (resulting in a planned dose of 200 cGy). After 100 days the mouse is sacrificed and the tumorous and non-tumorous sections of the brain are stained with hematoxylin and eosin for histologic examination. The tumor bearing and control brain tissue sections are compared.

Administration

The texaphyrin agents are administered at a therapeutically effective dosage, e.g., a dosage sufficient to provide treatment for the disease states previously described. The metellotexaphyrin to be used in the method of the invention will be administered in a pharmaceutically effective amount, employing a method of administration, and means of activation by neutron irradiation as is known in the art.

Dosages: The specific dose will vary depending on the particular metallotexaphyrin-chosen, the dosing regimen to be followed, employing dosages within the range of about 0.01 mg/kg/treatment up to about 50 mg/kg/treatment (depending on the molecular weight of the conjugate). It will be appreciated by one skilled in the art, however, that there are specific differences in the most effective dosimetry depending on the apical ligands chosen. Expected dose levels for an individual may range from about 1 mg/kg/treatment up to about 30 mg/kg/treatment or 0.5 μmol/kg to about 25 μmol/kg, depending on the texaphyrin chosen, administered in single or multiple doses (e.g. before each fraction of neutron irradiation).

Gadolinium texaphyrin is administered in a solution containing 2 mM, preferably in 5% mannitol, USP. Dosages of 0.6 mg/kg up to as high as 29.6 mg/kg have been delivered, preferably about 3.0 to about 15.0 mg/kg (for volume of about 90 to 450 mL) may be employed, optionally with pre-medication using anti-emetics above about 8.0 mg/kg. The texaphyrin is administered via intravenous injection over about a 5 to 15 minute period, followed by a waiting period of about 2 to 5 hours to facilitate intracellular uptake and clearance from the plasma and extracellular matrix prior to the administration of the neutron irradiation.

The administration of texaphyrin should occur before neutron irradiation or administration of the co-therapeutic agent. However, it should be noted that administration of the neutron irradiation could precede, follow, or occur without, the administration of the co-therapeutic agent.

The texaphyrin may be administered as a single dose, or it may be administered as two or more doses separated by an interval of time. Parenteral administration is typical, including by intravenous and interarterial injection. Other common routes of administration can also be employed, for example oral administration.

In general, the neutron beam must deliver neutrons that have an energy distribution sufficient to permit neutron capture at the target tissue. Specifically, the type of beam useful for neutron capture therapy is a beam of epithermal neutrons, i.e. neutrons possessing kinetic energies of between 0.4 eV and 10 keV. A neutron beam of this sort can be generated by a focused linear accelerator driven by a nuclear reactor or it can be from a synthetic nuclide Californium-252. The preferred method of generating a neutron beam is by a linear accelerator. Typically, a moderate filter for the neutron accelerator is needed to deliver thermal neutrons and higher energy epithermal neutrons. Commercially available linear accelerators delivering the epithermal beam may be employed in the practice of the invention. The diameter of the neutron beam can strongly influence the degree of penetration, and the diameter of the beam cross-section is preferably larger than the diameter of the tumor being irradiated. The preferred duration of treatment is a dose or fluence rate of about 5×10¹² n/cm²

Texaphyrins are provided as pharmaceutical preparations. A pharmaceutical preparation of a texaphyrin may be administered alone or in combination with pharmaceutically acceptable carriers, in either single or multiple doses. Suitable pharmaceutical carriers include inert solid diluents or fillers, sterile aqueous solution and various organic solvents. The pharmaceutical compositions formed by combining a texaphyrin of the present invention and the pharmaceutically acceptable carriers (including infusion and perfusion fluids) are then easily administered in a variety of dosage forms such as injectable solutions.

For parenteral administration, solutions of the texaphyrin in sesame or peanut oil, aqueous propylene glycol, or in sterile aqueous solution may be employed. Such aqueous solutions should be suitably buffered if necessary and the liquid diluent first rendered isotonic with sufficient saline, for example.

It has been discovered that texaphyrins have a tendency to aggregate in aqueous solution, which potentially decreases their solubility. Aggregation (self-association) of polypyrrolic macrocyclic compounds, including porphyrins, sapphyrins, texaphyrins, and the like, is a common phenomenon in water solution as the result of strong intermolecular van der Waals attractions between these flat aromatic systems. Aggregation may significantly alter the photochemical characteristics of the macrocycles in solution, which is shown by large spectral changes, decrease in extinction coefficient, etc.

It has been found that addition of a carbohydrate, saccharide, polysaccharide, or polyuronide to the formulation decreases the tendency of the texaphyrin to aggregate, thus increasing the solubility of the texaphyrin in aqueous media. Preferred anti-aggregation agents are sugars, in particular mannitol, dextrose or glucose, preferably mannitol, in about 2-8% concentration, more preferably about 5% concentration. These aqueous solutions are especially suitable for intravenous, intramuscular, subcutaneous and intraperitoneal administration. In this connection, the sterile aqueous media that can be employed will be known to those of skill in the art in light of the present disclosure.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy use with a syringe exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, cyclodextrin derivatives, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Activation Means

After the use of the compounds of the invention as neutron capture agents, it is often desirable to employ them as radiosensitizers or photodynamic sensitizers as a second step. That is, after administration of the compound of Formula I as described above, and its bombardment with neutrons, the compound remaining at the site of bombardment can then be further treated with a means of activation (through a therapeutic energy or agent) as is known in the art. The therapeutic energy or agent to be used includes photodynamic therapy, radiation sensitization, chemotherapy, and sonodynamic therapy. The compounds disclosed herein can also be used both diagnostically (e.g. magnetic resonance or fluorescence imaging to detect the presence of a disease) and therapeutically (to treat that disease).

### EXAMPLES

The following examples are given to enable those skilled in the art to more clearly understand and to practice the present invention. They should not be considered as limiting the scope of the invention, but merely as being illustrative and representative thereof.

### Example 1

### Preparation of ¹⁵⁷Gadolinium Texaphyrin

¹⁵⁷Gadolinium texaphyrin metal complex was prepared using standard methods previously described in Sessler et al., J. Phys. Chem., vol. 103, pgs. 787-794 (1999) and Young et al., Photochem. Photobiol., vol. 63, pgs. 892-897 (1996). The macrocyclic ligand, 9,10-diethyl-7,12-dihydro-20,21-bis[2-[2-(2-methoxyethoxy)ethoxy]ethoxy]-4,15-dimethyl-3,6:8,11:13,16-triimino-1,18-benzodiazacycloeicosine-5,14-dipropanol hydrochloride (IP-NP2, 2.2 g, 2.39 mmol), was oxidatively metallated using gadolinium(157) nitrate pentahydrate (1.02 g, 2.39 mmol) and triethylamine (3.3 mL, 23.9 mmol) in air-saturated methanol (200 mL) at reflux. After completion of the reaction (as judged by the optical spectrum of the reaction mixture), the deep green solution was cooled to room temperature and the solvent reduced to 150 mL under reduced pressure. The dark green solution was loaded onto a column (8 cm length×2.5 cm diameter) of pretreated Ambersep® 900 anion exchange resin (resin in the acetate form). The eluent containing the crude bis-acetate gadolinium (III) texaphyrin was collected, concentrated to dryness under reduced pressure, and dried in vacuo for 8 h. The resulting dark solid was suspended in acetone (60 mL), stirred for 30 min at room temperature, and then filtered to wash away the red/brown impurities (incomplete oxidation products and excess triethylamine). The crude complex (˜2 g) was dissolved into MeOH (60 mL), stirred for ˜30 min, and then filtered through celite into a 250 mL RBF. An additional 20 mL of MeOH and 8 mL of water were added to the flask along with acetic acid washed SAY-54 zeolite (10 g). The resulting mixture was agitated or shaken for 2 h, then filtered to remove the zeolite. The zeolite cake was rinsed with MeOH (100 mL) and the rinse solution added to the filtrate. The solvent was removed under reduced pressure and resulting dark solid dried in vacuo for 2 h. This solid was re-suspended in acetone (60 mL), stirred for 15 min, filtered, and vacuum dried for ˜18 h at 35-40° C. to afford 1.65 g (60%) of 4,5-diethyl-10,23-dimethyl-9,24-bis(3-hydroxypropyl)-16,17-bis[2-[2-(2 methoxyethoxy)ethoxy]ethoxy]pentaazapentacyclo[20.2.1.1^{3,6}.1^{8,11}.0^{14,19}]heptacos a-1,3,5,7,9,11(27),12,14,16,18,20,22(25),23-tridecaene bis acetate hydrate. FAB MS, [M-2OAc⁻]: m/e 1030; HRMS, [M-2OAc⁻]: m/e 1029.4054 (calcd. for [C₄₈H₆₆¹⁵⁷GdN₅O₁₀]²⁺, 1029.4049). Anal. calcd. for [C₄₈H₆₆GdN₅O₁₀](OAc)₂(H₂O)_{1/2}: C, 53.96; H, 6.36; N, 6.05; Gd, 13.59. Found: C, 53.94; H, 6.03; N, 5.81; Gd, 13.72.

### Example 2

### Determination of Neutron Capture Activity In Vitro Utilizing the V79 Chinese Hamster Cell Survival Assay

The effect of neutron irradiation on V79 Chinese hamster cells incubated for twelve hours with ¹⁵⁷Gd-Tex is determined by a modification of the procedure originally described by Fairchild et al., Cancer Research 50 (1990). Three sets of sample plates of V79 cells are prepared. The first set (control cells) are suspended with 30 ppm ¹⁵⁷Gd-Tex in growth medium. The second set (washed cells) are suspended with 30 ppm ¹⁵⁷Gd-Tex in growth medium, then washed 3 times with PBS, trypsinized and harvested with gadolinium from reagents prior to suspension. The third set (ambient cells) are suspended in a 30 ppm ¹⁵⁷Gd containing medium.

All of the cells are irradiated within 1-2 hours following suspension in growth medium at a population density of 3.0×10⁵ cells/ml. The neutron beam fluence rate at the center of the sample is 2.8×10¹¹ n/cm² min. All three of the cell samples are plated for undisturbed colony growth for 5-6 days, washed with PBS, fixed with buffered formalin and stained with Giemsa, prior to optoelectronic counting with an Artec colony counter.

The survival of the control cells is significantly higher than the survival of the washed or ambient cells.

### Example 3

### Determination of Neutron Capture Activity Utilizing Glioma 261 Cells

This procedure is a modification of a procedure initially described by Saris et al., Cancer Research, vol. 52 (1992). Glioma 261 tumor fragments are injected approximately 2 mm deep to the dura of the brain in 42 female C57BL/6 mice. The mice are randomized into 5 groups. The mice in group A receive ???? mg/kg Gd-Tex and 1.2×10¹² neutrons/cm² resulting in 20 Gy. Group B mice receive 1.2×10¹² neutrons/cm² resulting in 20 Gy. The mice in group C receive ???? mg/kg Gd-Tex and 10 Gy of photon irradiation. Group D receives ???? mg/kg of Gd-Tex alone. Group E receives no treatment.

After 5 days (when the tumor is approximately 2 mm), ???? mg/kg of a texaphyrin is administered by intravenous injection (groups A, C and D only). ???? Hours later, the mice in groups A and B are anesthetized and irradiated with a neutron beam of 1.2×10¹² neutrons/cm² to result in a planned total dose of 20 Gy. The mice in group C are anesthetized and irradiated with photons at a rate of 1.4±0.1 for a total dose of 10 Gy.

All of the mice are sacrificed by cervical dislocation when moribund or after 100 days. After sacrifice, the brains are removed and sections of the brain are taken from the anterior frontal cortex to the posterior temporal cortex and through the region of the intracerebral tumor. The sections of the brain are stained with hematoxylin and eosin for histologic examination. The sections of the brains are compared.

The animals in group A (receiving both Gd-Tex and neutron irradiation) have a greater survival rate than any of the other groups, indicating successful in vivo neutron capture treatment of the experimentally induced tumor.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

What is claimed is:

1. A method for treating a disease or condition in a mammal resulting from the presence of neoplastic tissue or an atheroma, which method comprises:
a) administering to a mammal in need of such treatment a therapeutically effective amount of a neutron capture agent of the formula: 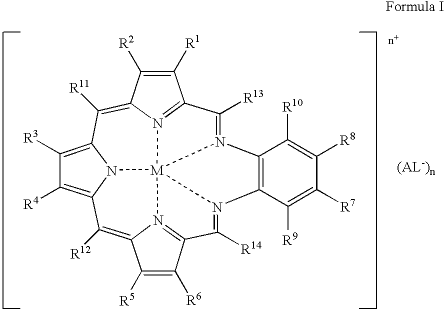
wherein:
AL is an apical ligand
M is a metal, an enriched metal, or a pure isotope thereof, having a neutron capture cross section greater than about 1,000 b;
n is an integer of 1-5;
R¹, R², R³, R⁴, R⁵, R⁶, R⁷, and R⁸ are independently chosen from the group consisting of hydrogen, halogen, hydroxyl, optionally substituted alkyl, optionally substituted alkenyl, optionally substituted alkynyl, optionally substituted aryl, optionally substituted heteroaryl, optionally substituted haloalkyl; nitro, acyl, optionally substituted alkoxy, optionally substituted amino, carboxyl, optionally substituted carboxyalkyl, optionally substituted carboxyamide, optionally substituted carboxyamidealkyl, optionally substituted cycloalkyl, optionally substituted arylalkyl, optionally substituted heteroarylalkyl, optionally substituted heterocycloalkylalkyl, and the group —X—Y, in which X is a covalent bond or a linker and Y is a catalytic group, a chemotherapeutic agent, or a site-directing molecule; and
R⁹, R¹⁰, R¹¹, R¹², R¹³ and R¹⁴ are independently hydrogen, optionally substituted alkyl, optionally substituted aryl, optionally substituted alkoxy, optionally substituted carboxyalkyl, or optionally substituted carboxyamidealkyl;
with the proviso that the halogen is other than iodide and the haloalkyl is other than iodoalkyl; and
b) irradiating the neoplastic tissue or the atheroma with a neutron beam.

2. The method of claim 1 wherein M is gadolinium.

3. The method of claim 3, wherein the gadolinium consists essentially of the isotope of gadolinium ¹⁵⁷Gd, or gadolinium enriched in the isotope ¹⁵⁷Gd.

4. The method of claim 4 wherein M is isotopically pure ¹⁵⁷Gd.

5. The method of claim 4, wherein R³ and R⁴ are ethyl, R¹ and R⁶ are methyl, and R⁹, R¹⁰, R¹¹, R¹², R¹³ and R⁴ are hydrogen.

6. The method of claim 6, wherein R² and R⁵ are 3-hydroxypropyl.

7. The method of claim 7, wherein R⁷ and R⁸ are 2-[2-[-(2-methoxyethoxy)ethoxy]ethoxy.

8. The method of claim 8, wherein the apical ligand is acetic acid.

9. The method of claim 9, wherein the disease or condition is a tumor.

10. The method of claim 9, wherein the disease or condition is arteriosclerosis.

11. The method of claim 1, further comprising treating the neoplastic tisssue or atheroma with a therapeutic energy means chosen from photoirradiation, ionizing radiation, and ultrasound, or a chemotherapeutic agent.

12. A method of neutron capture therapy comprising administering an effective amount of enriched ¹⁵⁷Gadolinium texaphyrin to a mammal in need thereof and providing an effective amount of neutron irradiation.

13. The method of claim 12 wherein the irradiation consists of administering 5×10¹² neutrons per cm².

14. The method of claim 13 wherein the target tissue to be treated is cancer tissue.

15. The method of claim 13 wherein the target to be treated is plaque.

16. A compound of the formula: 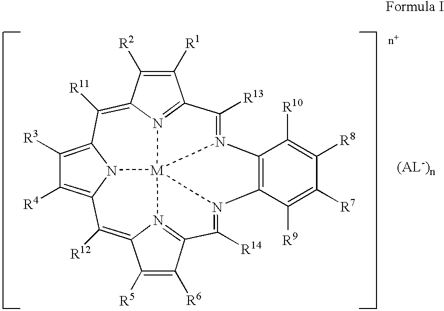
wherein:
AL is an apical ligand
M is an enriched metal, or a pure isotope thereof, having a neutron capture cross section greater than about 1,000 b;
n is an integer of 1-5;
R¹, R², R³, R⁴, R⁵, R⁶, R⁷, and R⁸ are independently chosen from the group consisting of hydrogen, halogen, hydroxyl, optionally substituted alkyl, optionally substituted alkenyl, optionally substituted alkynyl, optionally substituted aryl, optionally substituted heteroaryl, optionally substituted haloalkyl; nitro, acyl, optionally substituted alkoxy, saccharide, optionally substituted amino, carboxyl, optionally substituted carboxyalkyl, optionally substituted carboxyamide, optionally substituted carboxyamidealkyl, optionally substituted heterocycle, optionally substituted cycloalkyl, optionally substituted arylalkyl, optionally substituted heteroarylalkyl, optionally substituted heterocycloalkylalkyl, and the group —X—Y, in which X is a covalent bond or a linker and Y is a catalytic group, a chemotherapeutic agent, or a site-directing molecule; and
R⁹, R¹⁰, R¹¹, R¹², R¹³ and R¹⁴ are independently hydrogen, optionally substituted alkyl, optionally substituted aryl, optionally substituted alkoxy, optionally substituted carboxyalkyl, or optionally substituted carboxyamidealkyl;
with the proviso that the halogen is other than iodide and the haloalkyl is other than iodoalkyl.

17. The compound of claim 16 wherein M is gadolinium enriched in the isotope ¹⁵⁷Gd.

18. The compound, complex or salt of claim 17 wherein M is isotopically pure ¹⁵⁷Gd.

19. The compound of claim 18, wherein R³ and R⁴ are ethyl, R¹ and R⁶ are methyl, and R⁹, R¹⁰, R¹¹, R¹², R¹³ and R¹⁴ are hydrogen.

20. The compound of claim 19, wherein R² and R⁵ are 3-hydroxypropyl.

21. The compound of claim 20, wherein R⁷ and R⁸ are 2-[2-[-(2-methoxyethoxy)ethoxy]ethoxy.

22. The compound of claim 21, wherein the apical ligand is acetic acid.

23. A pharmaceutical composition comprising at least one pharmaceutically acceptable excipient and a therapeutically effective amount of a compound, complex or salt of claim 16.

24. The compound of claim 23 wherein M is gadolinium enriched in the isotope ¹⁵⁷Gd.

25. The compound of claim 24 wherein M is ¹⁵⁷Gd.

26. The compound of claim 25, wherein R³ and R⁴ are ethyl, R¹ and R⁶ are methyl, and R⁹, R¹⁰, R¹¹, R¹², R¹³ and R¹⁴ are hydrogen.

27. The compound of claim 26, wherein R² and R⁵ are 3-hydroxypropyl.

28. The compound of claim 27, wherein R⁷ and R⁸ are 2-[2-[-(2-methoxyethoxy)ethoxy]ethoxy.

29. The compound of claim 28, wherein the apical ligand is acetic acid.

30. The method of claim 2, wherein the neutron capture agent is co-administered with chemotherapeutic agent selected from: a platinum coordination complex, an anthracenedione, an anthracycline, a substituted urea, a methyl hydrazine derivative, or an adrenocortical suppressant.

31. The method of claim 30, wherein the chemotherapeutic agent is paclitaxel, etoposide, 4-OH cyclophosphamide, cisplatin, doxorubicin, or bleomycin.

32. The method of claim 2, wherein the neutron capture agent is co-administered with a photosensitizing agent.

33. The method of claim 32, wherein after co-administration,
a) the neoplastic tissue or the atheroma is irradiated with a neutron beam; and
b) the neoplastic tissue or the atheroma is treated with a therapeutic energy means.

34. The method of claim 33, wherein the photosensitizing agent is a metallotexaphyrin.

35. The method of claim 34 wherein the texaphyrin is motexafin lutetium.
